(19) United States
(12) Patent Application Publication
(10) Pub. No.: US 2023/0043018 A1
(43) Pub. Date: Feb. 9, 2023

(54) Smart ring for use with a user device and Wi-Fi network

(71) Applicant: Plume Design, Inc., Palo Alto, CA (US)

(72) Inventors: Crystal Wai, Redwood City, CA (US); Shuhan Liu, San Jose, CA (US); Hsiangyin Cheng, Taipei City (TW); Meng-Jung Chuang, Taipei City (TW); Liem Hieu Dinh Vo, San Jose, CA (US); Richard Chang, Los Altos, CA (US); Ming-Tsung Su, Taipei City (TW); Hao-Hsiu Huang, Taipei City (TW); Jeffrey ChiFai Liew, Millbrae, CA (US); Zhicheng Qiu, Cupertino, CA (US); Cuong Vu, Union City, CA (US); Fahri Diner, Zurich (CH); Miroslav Samardzija, Mountain View, CA (US); Shu Chun Shen, Taipei City (TW)

(21) Appl. No.: 17/714,220

(22) Filed: Apr. 6, 2022

Related U.S. Application Data
(63) Continuation-in-part of application No. 17/391,531, filed on Aug. 2, 2021.

(51) Int. Cl. H04W 4/029; H04W 52/02; H04W 4/38; G08B 25/01; A61B 5/00; A61B 5/11

(52) U.S. Cl. CPC H04W 4/029; H04W 52/0254; H04W 4/38; G08B 25/016; A61B 5/6826; A61B 5/1117; H04W 84/12

(57) ABSTRACT

A smart ring includes a battery, memory, processing circuitry, a plurality of sensors, a plurality of antennas, and a battery, each coupled to one another and all enclosed in a casing, wherein the processing circuitry is configured to conserve the battery by any of sending data to the cloud service when an application is open on the user device, sending data to the cloud service when a threshold is crossed, waking up processing or communicating when there is a change in motion detected by the accelerometer.

### CROSS-REFERENCE TO RELATED APPLICATION(S)

The present disclosure is a continuation-in-part of U.S. patent application Ser. No. 17/391,531, filed Aug. 2, 2021, the contents of which are incorporated by reference in their entirety.

### FIELD OF THE DISCLOSURE

The present disclosure generally relates to a smart ring. More particularly, the present disclosure relates to systems and methods for smart ring for use with a user device and Wi-Fi network.

### BACKGROUND OF THE DISCLOSURE

Recently, “smart rings” have been developed and have become a popular consumer electronic device. From the outside, smart rings appear to be regular decorative rings. However, these smart rings may include wireless capabilities that allow them to pair with corresponding POS device for making payments. Also, some smart rings may instead be configured to pair with a smart phone. The wireless capabilities of these various smart rings require that antennas be incorporated into the ring. However, since some smart rings may be made of metal, it can be challenging to design and integrate antennas into the metallic rings. Typical designs on the market use chip antennas which require dedicated antenna volume that may already be scarce. Normally, these chip antennas have low performance as they typically rely on ground currents of very small Printed Circuit Board (PCB). Therefore, there is a need in the field of POS devices and smart rings to improve wireless communication with external devices.

Also, various conventional applications for smart rings include security (access control), payments, activity tracking, and feedback to a wearer. What has not yet been explored is integration of a smart ring with a mobile device, Wi-Fi network, and/or cloud service for a variety of applications. For example, people are living longer and seeking ways to maintain independence as they age, i.e., these people can be referred to as “active boomers.” Active boomers face challenges living at home in security, isolation, safety, and health. For security, active boomers can fall victim to scams, home intrusions, etc. For isolation, accidents or health degradation oftentimes go undetected. For safety, falls can occur. Finally, for health, obesity, hypertension, chronic obstructive pulmonary disease, Diabetes, Depression/Anxiety, Dementia are common health concerns. Monitoring and remaining active are key to preserving one's wellness

### BRIEF SUMMARY OF THE DISCLOSURE

The present disclosure relates to systems and methods for smart ring for use with a user device and Wi-Fi network. In an embodiment, a smart ring includes a battery, memory, processing circuitry, a plurality of sensors, a plurality of antennas, and a battery, each coupled to one another and all enclosed in a casing, wherein the processing circuitry is configured to conserve the battery by any of sending data to the cloud service when an application is open on the user device, sending data to the cloud service when a threshold is crossed, waking up processing or communicating when there is a change in motion detected by the accelerometer. The processing circuitry can be further configured to cause the smart ring to pair with any of a user device and a Wi-Fi access point in a distributed Wi-Fi system, and obtain measurements from any of the plurality of sensors and provide the measurements to a cloud service, via the any of the user device and the Wi-Fi access point.

The plurality of antennas can include an antenna for Bluetooth and Wi-Fi and an antenna for near field communication. The plurality of antennas can include an antenna for Bluetooth, wherein the antenna for Bluetooth is utilized in combination with the distributed Wi-Fi system to track a location of a wearer. The tracking of location of a wearer can be used for one or more of counting bathroom trips, counting kitchen trips, counting time spent in bed. The plurality of sensors can include a Photoplethysmography (PPG) sensor and an accelerometer. The PPG sensor can be configured to measure oxygen saturation. The accelerometer can be configured to detect motion and falls. The ring can include a plurality of a microphone, a speaker, haptic feedback, or a tap sensor. The ring can contact caregivers or emergency personnel based on input from one or more of the microphone or tap sensor.

In another embodiment, a cloud system include one or more processing devices comprising processors and memory storing instructions that, when executed, cause the processors to communicate with a smart ring containing a plurality of sensors and a plurality of antennas for measurements, wherein the communication with the smart ring saves power on the smart ring by communicating only when one or more of an application is open on a user device, a threshold is crossed, or there is a change in motion detected by an accelerometer in the ring. The instructions that, when executed, can further cause the processors to communicate with any of a user device and a Wi-Fi access point in a distributed Wi-Fi system, the any of the user device and the Wi-Fi access point is paired to the smart ring, and obtain the measurements from the memory in the smart ring, via the any of the user device and the Wi-Fi access point.

The instructions that, when executed, can further cause the processors to aggregate data from an at least first and second smart ring, such that a wearer wears one of the smart ring and the second smart ring at a time while another is charged. The measurements can be combined with other measurements taken by the distributed Wi-Fi system. The other measurements can include one or more of network usage, application usage, types of devices used.

The communication with the ring can include data from one or more of an accelerometer, a Photoplethysmography (PPG) sensor, a microphone, or a tap sensor. The plurality of antennas can include an antenna for Bluetooth, wherein the antenna for Bluetooth is utilized in combination with a distributed Wi-Fi system including the Wi-Fi access point to track a location of a wearer. The instructions that, when executed, can further cause the processors to combine the measurements with cohorts for comparison thereof. The instructions that, when executed, can further cause the processors to provide a notification to a third party based on a threshold being crossed. The instructions that, when executed, can further cause the processors to track a routine of a wearer based on the measurements. The instructions that, when executed, can further cause the processors to provide a Graphic User Interface (GUI) to display trends related to the measurements. The instructions that, when executed, can further cause the processors to detect issues based on the measurements and present notifications based thereon. A portion of the data communicated can be provided to a third party that supplies third party services. The third party services can include one or more of Meditation, Breath Training, Yoga, Diet, Exercise, Counseling, Healthcare.

In an embodiment, a wearable ring includes an inner surface and an outer surface; a first antenna component and a second antenna component, each disposed between the inner surface and the outer surface; a first electrical circuit connecting a first end portion of the first antenna component with a first end portion of the second antenna component; and a second electrical circuit connecting a second end portion of the first antenna component with a second end portion of the second antenna component, and wherein, based on configuration of the first electrical circuit and the second electrical circuit, the first antenna component and second antenna component are configured to operate in a given frequency band. The given frequency band can be one of a first frequency band and a second frequency band. Operation within the first frequency band can enable pairing with a user device and operation within the second frequency band can enable pairing with a Point of Sale (POS) device. The first frequency band can include one or more channels in a Bluetooth frequency band ranging from about 2.4000 GHz to about 2.4835 GHz and the second frequency band includes one or more channels in a Near-Field Communication (NFC) frequency band ranging from about 12.66 MHz to about 14.46 MHz

The configuration can include one of a dipole antenna arrangement and a loop antenna arrangement. The dipole antenna arrangement can be for Bluetooth and/or Wi-Fi and the loop antenna arrangement is for Near-Field Communication (NFC). The outer surface can include an outer shell having characteristics configured for parasitic reflection of transmission signals. The wearable ring can further include a battery configured to power one or more of the first and second electrical circuits, wherein the battery includes an outer metal casing that forms at least a portion of the first antenna component. The wearable ring can further include a Near-Field Communication (NFC) charger configured to create a magnetic field for charging the battery. The battery can serve as one of more of a ground plane, one of arms for a dipole antenna arrangement, and a current path for a loop antenna arrangement. The second antenna component can include at least a flexible printed circuit board on which at least a portion of the second electrical circuit resides. The second electrical circuit can include blocking elements, matching circuit elements, and transceiver elements to enable operation within either a first frequency band or a second frequency band. The first electrical circuit can include a choke inductor that behaves like an open circuit when operating within a first frequency band and behaves like a short circuit when operating within a second frequency band. The wearable ring can further include one or more of a conductive strip and a ferrite strip attached to one or more of the first and second antenna component.

### BRIEF DESCRIPTION OF THE DRAWINGS

The present disclosure is illustrated and described herein with reference to the various drawings, in which like reference numbers are used to denote like system components/method steps, as appropriate, and in which:

FIG. 1 is a diagram illustrating a system where a smart ring can wirelessly communicate with a POS machine and a mobile device, according to various embodiments of the present disclosure.

FIG. 2 is a diagram illustrating a cross-sectional view of a smart ring, according to various embodiments of the present disclosure.

FIG. 3 is a schematic diagram illustrating electrical circuitry of the smart ring of FIG. 2, according to various embodiments.

FIG. 4 is a diagram illustrating operation of the smart ring of FIG. 2 within a first frequency band, according to various embodiments.

FIG. 5 is a diagram illustrating operation of the smart ring of FIG. 2 within a second frequency band, according to various embodiments.

FIG. 6 is a diagram illustrating currents in a dipole antenna arrangement with parasitic currents, according to various embodiments.

FIG. 7 is a diagram illustrating currents in the dipole antenna arrangement shown in FIG. 6 with the antenna elements laid out linearly, according to various embodiments.

FIG. 8 is a graph illustrating the scattering parameter (S parameter) of S11 versus frequency of a matching circuit for use in the higher frequency band, according to various embodiments.

FIG. 9 is a diagram illustrating operation of the smart ring of FIG. 2, according to various embodiments.

FIG. 10 is a graph illustrating the S parameter S11 versus frequency of a choke inductor for use in the higher frequency band, according to various embodiments.

FIG. 11 is a diagram illustrating operation of the smart ring of FIG. 2 for operation in the second frequency band, according to various embodiments.

FIG. 12 is a diagram illustrating a side cross-sectional view of the smart ring of FIG. 2 and corresponding magnetic field lines, according to various embodiments.

FIG. 13 is a diagram illustrating a cross-sectional view of the smart ring of FIG. 2 and corresponding magnetic fields, according to various embodiments.

FIG. 14 is a diagram illustrating currents in a loop antenna arrangement with parasitic currents, according to various embodiments.

FIG. 15 is a diagram illustrating the smart ring of FIG. 2 with a strip of conductor film formed on one surface of a battery casing, according to various embodiments.

FIG. 16 is a diagram illustrating the smart ring of FIG. 2 with the strip of conductor film of FIG. 15 and strips of ferrite sheets formed on surfaces of the battery casing and a Flexible Printed Circuit (FPC), according to various embodiments.

FIG. 17 is a photograph illustrating a side view of a Near-Field Communications (NFC) charger for charging the smart ring of FIG. 2, according to various embodiments.

FIG. 18 is a photograph illustrating a top view of the NFC charger of FIG. 17, according to various embodiments.

FIG. 19 is a graph illustrating the S parameter of S21 versus frequency of the NFC charger for operation within the second frequency band, according to various embodiments.

FIG. 20 is a diagram illustrating an end portion of a battery casing used as one antenna element, according to various embodiments.

FIG. 21 is a photograph illustrating the end portion of the battery casing of FIG. 20, according to various embodiments.

FIGS. 22-24 are diagrams illustrating connections between the battery casing of FIG. 20 and an end portion of a FPC used as another antenna element, according to various embodiments.

FIG. 25 is a diagram illustrating an isometric, partially cut-away view of the smart ring of FIG. 2, according to various embodiments.

FIG. 26 is a diagram illustrating a cross-section side view of the smart ring of FIG. 2, according to various embodiments.

FIG. 27 is a photograph illustrating connections from positive and negative terminals of a battery to another end portion of the FPC, according to various embodiments.

FIG. 28 is a diagram of a smart ring.

FIG. 29 is a network diagram of a distributed Wi-Fi system with control via a cloud service.

FIG. 30 is a block diagram of a user device, e.g., a mobile device.

FIG. 31 is a diagram of a location with access points distributed therein, in different rooms.

FIG. 32 is screenshots of a Graphical User Interface (GUI) from the application illustrating management of the distributed Wi-Fi system.

FIGS. 33A, 33B, and 33C are screenshots of the GUI from the application illustrating management of the smart ring.

FIG. 34 is a screenshot of the GUI from the application illustrating location and motion detection.

FIG. 35 is a diagram of integration of third-party devices with the cloud service.

FIG. 36 is a diagram of a use case where a doorbell camera is integrated with the cloud service and the application to alert a specific person is at the door.

### DETAILED DESCRIPTION OF THE DISCLOSURE

In various embodiments, the present disclosure relates to systems and methods for smart ring for use with a user device and Wi-Fi network. A smart ring includes a battery, memory, processing circuitry, a plurality of sensors, a plurality of antennas, and a battery, each coupled to one another and all enclosed in a casing, wherein the processing circuitry is configured to cause the smart ring to pair with any of a user device and a Wi-Fi access point in a distributed Wi-Fi system, and obtain measurements from any of the plurality of sensors and provide the measurements to a cloud service, via the any of the user device and the Wi-Fi access point.

In another embodiment, the present disclosure is directed to antenna systems and circuitry, which may be embedded in a wearable device, such as a ring that may be worn on a wearer's finger. According to one implementation, an antenna system includes a first antenna component having a first end portion and a second end portion a second antenna component also having a first end portion and a second end portion. The antenna system also includes a first electrical circuit connecting the first end portion of the first antenna component with the first end portion of the second antenna component and a second electrical circuit connecting the second end portion of the first antenna component with the second end portion of the second antenna component. In response to the first and second electrical circuits being configured in a first state, the first antenna component and second antenna component are configured to operate within a first frequency band. In response to the first and second electrical circuits being configured in a second state, the first antenna component and second antenna component are configured to operate within a second frequency band.

### Antenna for a Smart Ring

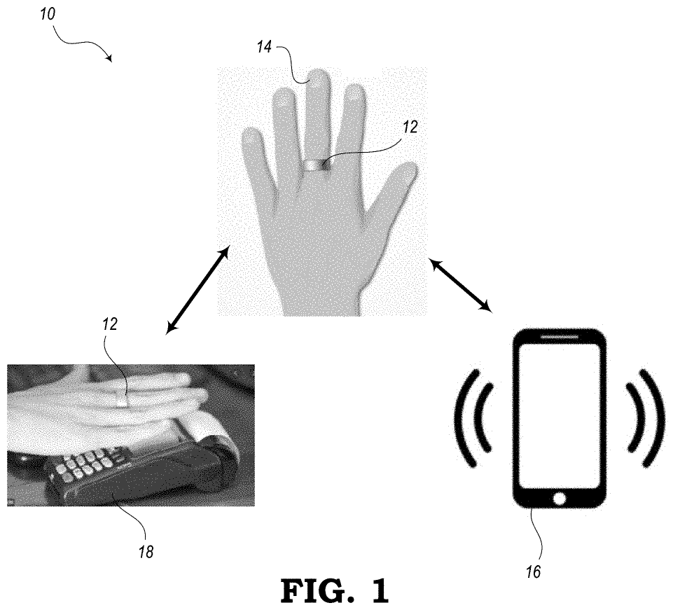

FIG. 1 is a diagram illustrating a system 10 in which a smart ring 12 can wirelessly communicate at short range to various devices. For example, the smart ring 12 may be worn on a finger 14 of a user (e.g., customer). When positioned near a mobile device 16, the smart ring 12 and mobile device 16 may be configured to operate within a first frequency band (e.g., Bluetooth frequencies) to enable communication therebetween. When positioned close to a Point-of-Sale (POS) machine 18, the smart ring 12 and POS machine 18 may be configured to operate within a second frequency band (e.g., Near Field Communication (NFC) frequencies) to enable communication therebetween.

Conventional smart rings normally do not allow operation within two separate frequency bands. However, according to the various embodiments of the present disclosure, various antenna components of the smart ring 12 include specific physical characteristics and electrical circuitry that enable operation at two different frequency band. This allows the smart ring 12 to pair with the mobile device 16 to enable operation within the first frequency band (e.g., Bluetooth) while also allowing the smart ring 12 to pair with the POS machine 18 to enable operation within the second frequency band (e.g., NFC). In particular, antenna portions, as described below, may be configured to be fully embedded in a normal-sized ring. These antenna portions may include, for example, the electrically conductive battery casing and also a conductive trace or film on a Flexible Printed Circuit (FPC) or other suitable flexible board that can be embedded within the normal-sized ring. By using these components, which may already be needed for wireless communication, it may be possible to minimize the extra number of parts and circuitry to conserve space within the outer shell of the smart ring 12.

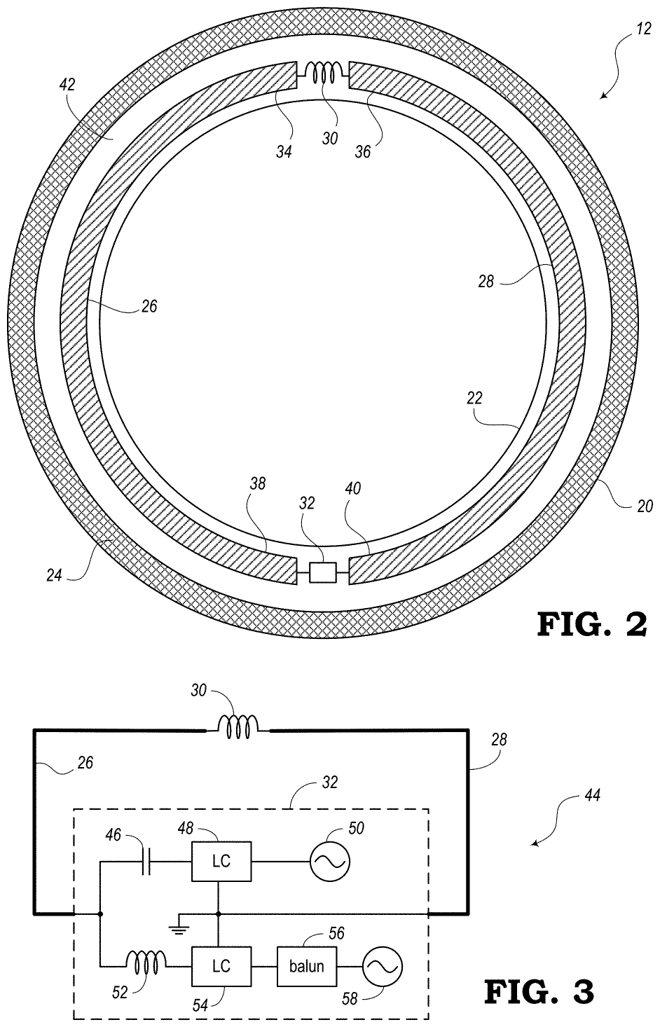

FIG. 2 shows a cross-sectional view of an embodiment of the smart ring 12. The smart ring 12 includes an outer surface 20 that may usually be visible when it is worn on a user's finger 14 (not shown in FIG. 2) and an inner surface 22 that may usually be in contact with the user's finger 14. An outer portion of the smart ring 12 may include a metallic layer 24, which may include the outer surface 20 in some embodiments.

Also, the smart ring 12 includes a first antenna component 26 and a second antenna component 28. The first and second antenna components 26, 28, in combination, may form a ring or tube having a relatively narrow width (e.g., measured from an outer surface to an inner surface as shown in FIG. 2) and a relatively narrow depth (e.g., measured into the page). In some embodiments, the depth of each of the first and second antenna components 26, 28 may have a dimension that is greater than its width.

Furthermore, the smart ring 12 includes a first electrical circuit 30 and a second electrical circuit 32. The first electrical circuit 30 is configured to electrically connect a first end portion 34 of the first antenna component 26 with a first end portion 36 of the second antenna component 28. Also, the second electrical circuit 32 is configured to electrically connect a second end portion 38 of the first antenna component 26 with a second end portion 40 of the second antenna component 28.

FIG. 3 is a schematic diagram illustrating an embodiment of an antenna circuit 44. In this embodiment, the antenna circuit 44 includes the first electrical circuit 30, the second electrical circuit 32, and the first and second antenna components 26, 28 connected between the first and second electrical circuits 30, 32. According to some embodiments, the first electrical circuit 30 may simply include an inductor configured to act like an open circuit at higher frequencies (e.g., Bluetooth and Wi-Fi frequencies) and act like a short circuit at lower frequencies (e.g., NFC frequencies).

As shown in the embodiment of FIG. 3, the second electrical circuit 32 includes a first set of components 46, 48, 50 configured for operation at the higher frequency range (e.g., Bluetooth, Wi-Fi) and a second set of components 52, 54, 56, 58 configured for operation at the lower frequency range (e.g., NFC). The first set of components includes a frequency blocking device 46 (e.g., series-connected capacitor), a higher-frequency matching circuit 48 (e.g., a combination of series-connected and shunt-connected inductors and capacitors), and a higher-frequency radio transceiver 50. The second set of components includes a higher-frequency choke or choke inductor 52 (e.g., a series-connected inductor or ferrite bead), a lower-frequency matching circuit (e.g., combination of series-connected and shunt-connected capacitors), a lower-frequency balun 56, and a lower-frequency radio transceiver 58. The matching circuits 48, 54 may be connected to ground and the radio transceivers 50, 58 may also be connected to ground.

To design an efficient antenna according to antenna theory, the length of the antenna is typically one fourth, one half, or one whole wavelength of the frequency of operation. For example, at a Bluetooth or Wi-Fi frequency of about 2.4 GHz, the wavelength is about 120 mm. At an NFC frequency of about 13.56 MHz, the wavelength is about 22 m (i.e., 22,000 mm). Other similar wavelengths may be applicable at other Bluetooth or Wi-Fi frequencies (e.g., about 2.4000 GHz to about 2.4835 GHz) or at other NFC frequencies (e.g., about 12.66 MHz to about 14.46 MHz).

Rings typically vary in diameter from about 12 mm to about 22 mm and typically vary in internal circumference from about 49 mm to about 72 mm. Even the largest ring sizes are well below the typically minimum required diameter dimension of one-fourth of the wavelength (i.e., 120 mm/4=30 mm at Bluetooth frequency). Even if the entire ring is used for antenna volume it still would not be enough. This does not even include all the other parts, like battery, photo diode sensors, RF board, chips, etc.

Typical designs on the market use chip antennas that are a few mm by a few mm in size, but which require dedicated antenna volume that is already scarce. In addition, chip antennas have low performance as they typically rely on PCB ground currents that are weak in ring size (e.g., due to the small size of the PCB itself). Nevertheless, the configuration of the first and second antenna components 26, 28 as described with respect to the embodiments of the present disclosure allows the circumference dimension to be utilized in a specific way to enable operation in both frequency bands. Operation is contemplated in both frequency bands simultaneously. For example, the NFC band could be used for charging while the Bluetooth band is used for accessing another Bluetooth device, e.g., a phone, or Wi-Fi access point. Another example can include using the ring for payment (NFC) while maintaining a connection to a phone (Bluetooth).

Therefore, according to various implementations of the present disclosure, antenna systems and antenna circuits are provided. In one example, an antenna system may include the first antenna component 26 having a first end portion 34 and a second end portion 38 and the second antenna component 28 having a first end portion 36 and a second end 40. The antenna system may also include the first electrical circuit 30 connecting the first end portion 34 of the first antenna component 26 with the first end portion 36 of the second antenna component 28 and a second electrical circuit 32 connecting the second end portion 38 of the first antenna component 26 with the second end portion 40 of the second antenna component 28. In response to the first and second electrical circuits 30, 32 being configured in a first state, the first antenna component 26 and second antenna component 28 are configured to operate within a first frequency band (e.g., Bluetooth, Wi-Fi). In response to the first and second electrical circuits 30, 32 being configured in a second state, the first antenna component 26 and second antenna component 28 are configured to operate within a second frequency band (e.g., NFC).

Also, in response to the first and second electrical circuits 30, 32 being configured in the first state, the first antenna component 26 and second antenna component 28 are configured in a dipole antenna arrangement (e.g., when the inductor 30 acts as an open circuit). In response to the first and second electrical circuits 30, 32 being configured in the second state, the first antenna component 26 and second antenna component 28 are configured in a loop antenna arrangement (e.g., when the inductor 30 acts as a short circuit). According to some embodiments, the antenna system may be incorporated in a wearable device, such as a ring or smart ring 12, which may be worn on a finger of the wearer. The ring 12 may include an outer shell (e.g., metallic layer 24) having characteristics configured for parasitic reflection of transmission signals.

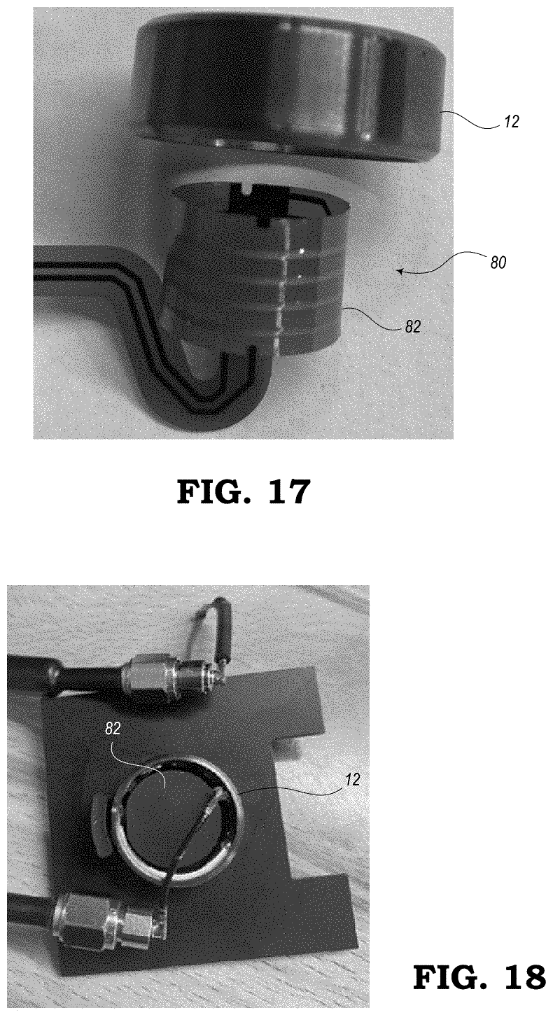

According to some embodiments, operation within the first frequency band may enable pairing with a smart phone (e.g., mobile device 16) and operation within the second frequency band enable pairing with a Point of Sale (POS) device (e.g., POS machine 18). The antenna system may further include a battery configured to power one or more of the first and second electrical circuits 26, 28. The battery may include an outer metal casing that forms at least a portion of the first antenna component 26. The antenna system may also include a Near-Field Communication (NFC) charger (described with respect to FIGS. 17-19). The NFC charger may be configured to create a magnetic field for charging the battery. The first frequency band may include one or more channels in a Bluetooth or Wi-Fi frequency band ranging from about 2.4000 GHz to about 2.4835 GHz and the second frequency band may include one or more channels in a Near-Field Communication (NFC) frequency band ranging from about 12.66 MHz to about 14.46 MHz

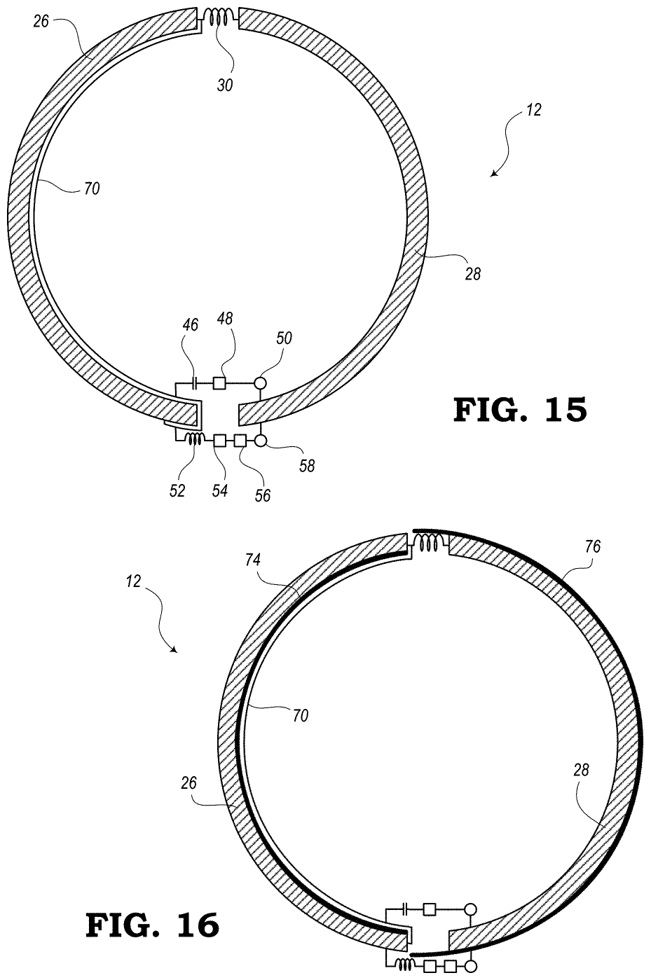

The second antenna component 28 may include at least a Flexible Printed Circuit (FPC) or FPC board on which at least a portion of the second electrical circuit 28 resides. The first electrical circuit 30 may include a choke inductor that behaves like an open circuit when operating within the first frequency band and behaves like a short circuit when operating within the second frequency band. The second electrical circuit 32 may include blocking elements 46, 52, matching circuit elements 48, 54, and transceiver elements 50, 58 to enable operation within either the first frequency band or second frequency band. Also, according to embodiments described with respect to FIGS. 15 and 16, the antenna system may further include one or more conductive strips and/or one or more ferrite strips attached to one or more of the first and second antenna components 26, 28.

In operation, the smart ring 12 uses the metal jacket or casing on the battery as part of the first antenna component 26 and can therefore serve as one of the arms of a dipole-like antenna, radiator, or transceiver. When the first electrical circuit 30 is shorted, the battery casing can serve as part of a current path for a loop antenna including both antenna components 26, 28. The battery can also serve as the ground plane of the antenna. In some embodiments, a thin metallic film (e.g., copper tape) can be installed along an outside surface of the battery (e.g., as described below with respect to FIGS. 15 and 16).

The antenna may include, at least partially, one or more traces on the FPC board or PCB (i.e., flexible or rigid boards). Other parts of the antenna may include, at least partially, the metallization on the outside of the battery (e.g., battery case). A ground plane of the FPC may be the actual radiating element of the antenna, (e.g., no separate trace for the antenna element). Various techniques may be applied to protect the electronics from potentials that might be induced in the ground plane, disrupting their operation.

For the higher-frequency (Bluetooth, Wi-Fi) operation, the antenna has a dipole arrangement, but for the lower-frequency (NFC) operation, the antenna has a loop arrangement. The dipole can approximate a half wave dipole considering loading and tuning. The creation of either the dipole or loop arrangement can be determined by the state of the choke inductor 30. Also, the choke inductor 30 enables the antenna circuit to include higher-frequency or lower-frequency arrangements that can be tuned independently.

The metallic layer 24 of the smart ring 12 can be a parasitic element with a predetermined thickness. Also, the smart ring 12 may include a gap 42 between the metallic layer 24 and the first and second antenna components 26, 28. The gap 42 may have a predetermined width that can be designed to control the parasitic characteristics of the metallic layer 24.

The second electrical circuit 32 may include the capacitor 46 configured for isolation to protect the higher frequencies from the lower frequencies. Also, isolation by the inductor 52 can protect the lower frequency (NFC) circuits from the higher frequency signals.

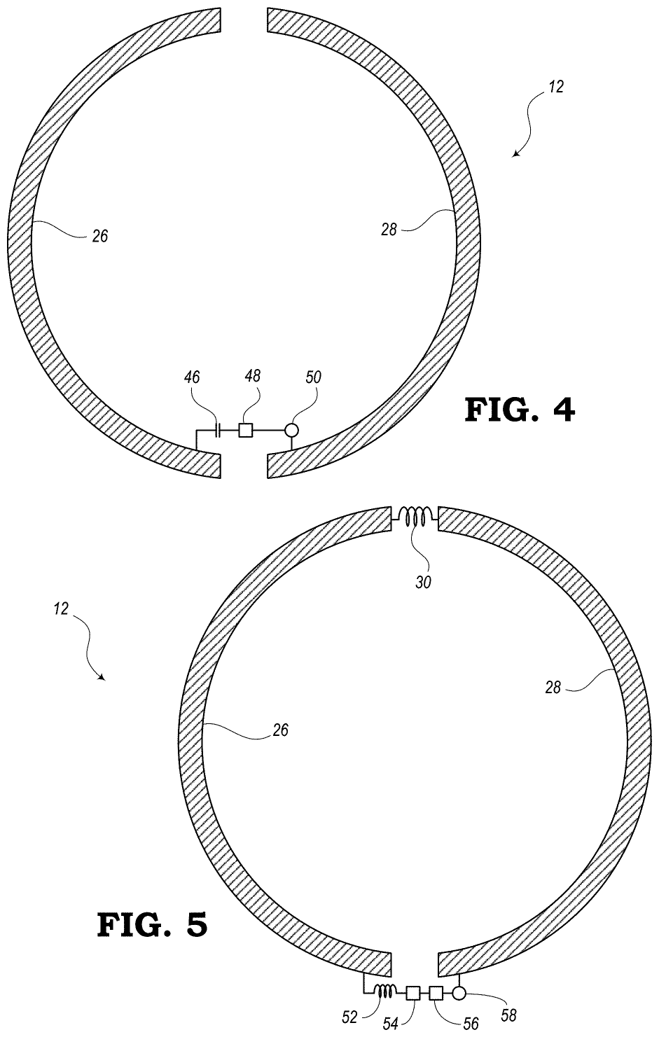

FIG. 4 is a diagram illustrating operation of the smart ring 12 within a first (higher) frequency band, according to some embodiments. In the higher frequency operation (e.g., frequency band of about 2.0 GHz to about 2.4 GHz), the choke inductors 30, 52 are “open.” As a result, the antenna circuitry (e.g., first and second antenna components 26, 28) effectively become a folded dipole device where a first arm includes the first antenna component 26 and a second arm includes the second antenna component 28. Also, the bottom portion of the second electrical circuit 32, which includes the components 52, 54, 56, 58, are essentially isolated as a result of the inductor 52 acting as an open circuit. Again, the first antenna component 26 may include the battery and/or battery casing and the second antenna component 28 may include the FPC, surrounded by the parasitic element (e.g., metallic layer 24, not shown in FIG. 4).

In the arrangement of FIG. 4, the smart ring 12 is configured for higher frequency (e.g., Bluetooth, Wi-Fi) operation. Accordingly, the applicable wavelengths (e.g., carrier frequency wavelengths) may be about 120 mm at a frequency of about 2.4 GHz. The ring circumference may typically be about 50-70 mm, which is in neighborhood of a half wavelength. The battery casing and FPC can be about 25-35 mm long, which is in the neighborhood of a quarter wavelength. High frequency matching and chokes can be used to offset for embodiments in which the dipole arms are not exactly a quarter wavelength. At high frequency, the chokes are “open,” and a folded dipole antenna structure is created.

FIG. 5 is a diagram illustrating operation of the smart ring 12 within the second (lower) frequency band. In the lower frequency band (e.g., NFC, about 13.56 MHz), the capacitor 46 (e.g., NFC blocker) is “open” and the antenna circuit effectively becomes a loop antenna made up of the battery or battery casing (e.g., first antenna component 26) and the FPC (e.g., second antenna component 28). Also, the inductor 30 may act essentially like a short circuit at the lower frequencies. The loop antenna is surrounded by parasitic element (e.g., metallic layer 24, not shown in FIG. 5).

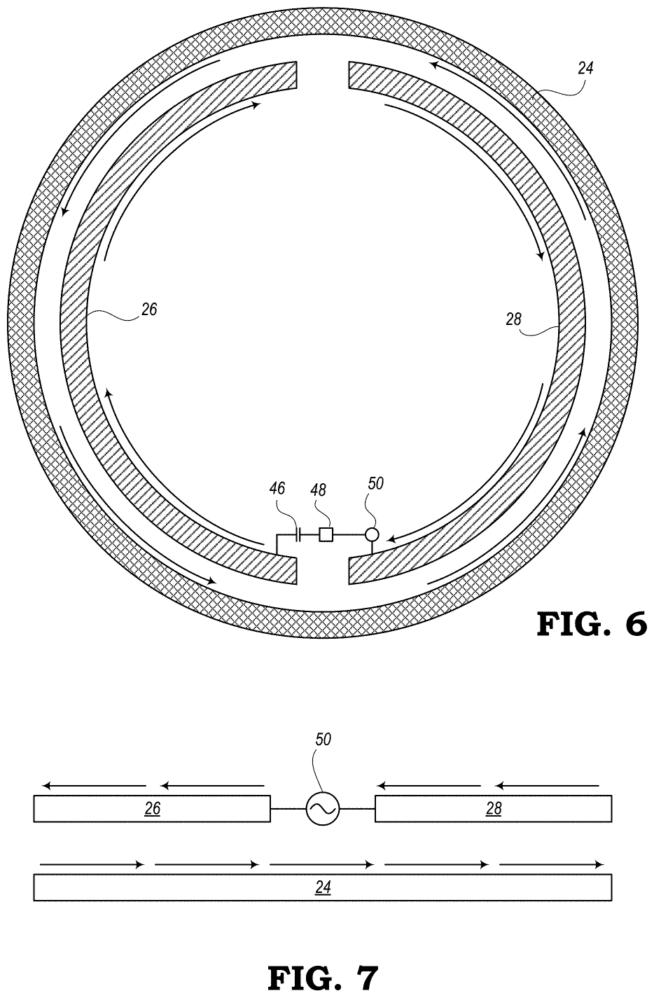

FIG. 6 is a diagram illustrating currents in the dipole antenna arrangement as shown in FIG. 4. Also, parasitic currents through the metallic layer 24 are shown. FIG. 7 is a diagram illustrating currents in the dipole antenna arrangement, where the antenna elements are laid out linearly.

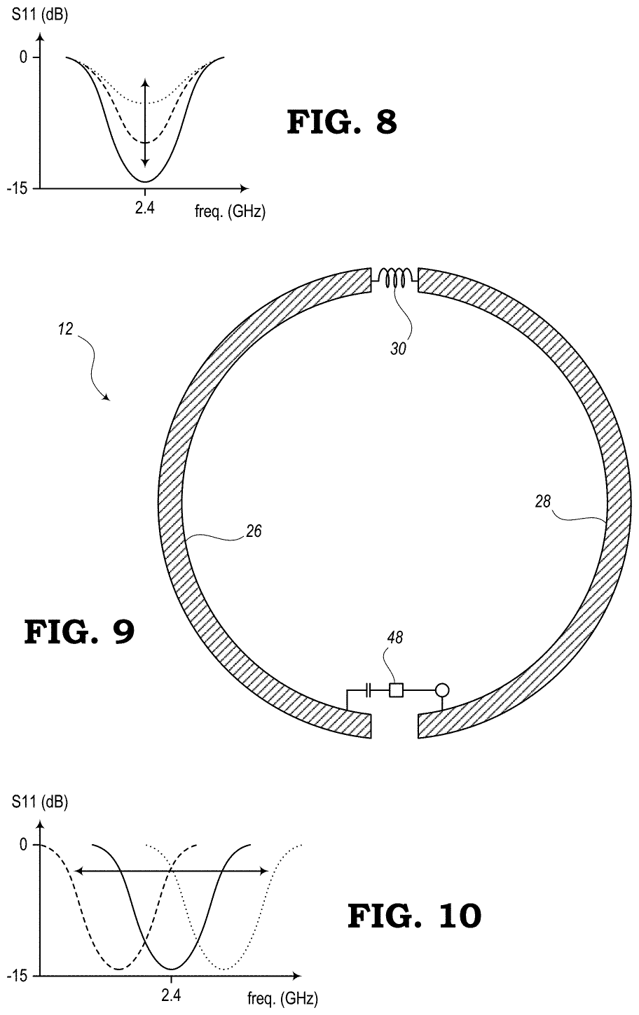

FIG. 8 is a graph illustrating the scattering parameter (S parameter) of S11 versus frequency of the matching circuit 48, as shown in FIGS. 3 and 9, for use in the higher frequency band. Inductance may be on the order of nH and capacitance may be on the order of pF. The term S11 may represent the input port voltage reflection coefficient of the scattering parameter matrix. FIG. 10 is a graph illustrating the S parameter S11 versus frequency of a choke inductor (e.g., inductor 30 shown in FIGS. 2, 3, and 9) for use in the higher frequency band. The inductor 30 may include an inductance on the order of μH. For the matching circuit 48, the inductance may be on the order of nH and the capacitors may have a capacitance on the order of pF.

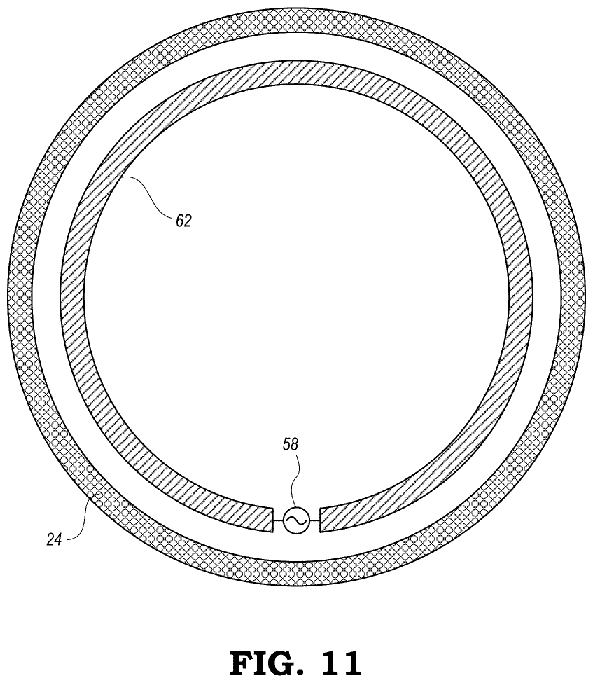

FIG. 11 is a diagram illustrating operation of the smart ring 12 for operation in the second (lower) frequency band when configured as a loop antenna 62 as shown in FIG. 5. The lower (NFC) band allows the smart ring 12 to operate at about 13.56 MHz, plus or minus about 0.9 MHz and utilizing the transceiver 58. In the NFC band, the wavelength is about 22 m (i.e., 22,000 mm). The battery (e.g., first antenna component 26) and the FPC (e.g., second antenna component 28) are effectively connected through the higher frequency choke (e.g., inductor 30) at about 13.56 MHz. The lower frequency antenna may have low resistance and high inductance in the loop (e.g., about 0.1 to about 3.0 micro Henries (μH)). The other higher frequency choke (e.g., inductor 52) in addition to the inductor 30 can also be used to offset a lack of inductance in the loop 62.

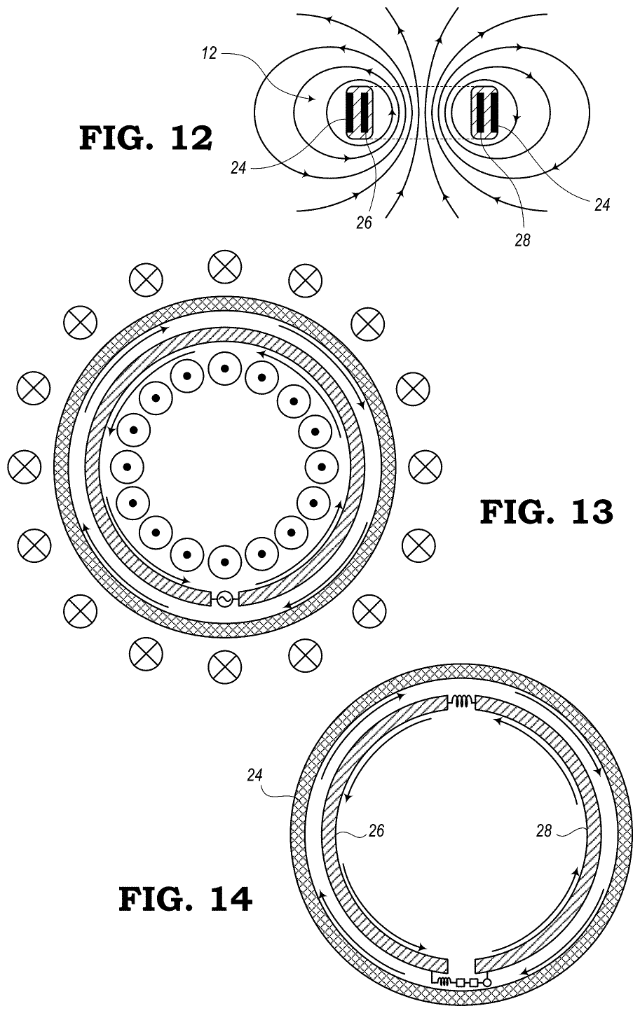

FIG. 12 is a cross-sectional view of the smart ring 12 from a side perspective, where the smart ring 12 extends orthogonally with respect to the page. In this embodiment, the metallic layer 24 is shown only at an outer portion of the smart ring 12, but, in other embodiments, the metallic layer 24 may extend around the entire periphery of the ring surface or partially around the periphery. FIG. 12 also shows a cross section of the first antenna component 26 (e.g., battery casing) and a cross section of the second antenna component 28 (e.g., FPC). Although the cross section of the first and second antenna components 26, 28 are shown as being rectangular, it should be understood that they may include any suitable shape for operation within the range of different sizes and configurations of various rings. Also shown in FIG. 12 are corresponding magnetic field lines based on radiation patterns of the transceivers 50, 58.

FIG. 13 is a cross-sectional view of the smart ring 12 from a top perspective, where the smart ring 12 is parallel to the page and the magnetic field lines extend orthogonally with respect to the page. For example, the circles with dots represent a direction of the magnetic field coming out of the page and circles with X_{S} represent a direction of the magnetic field going into the page. Also, the arrows in the counter-clockwise direction represent the direction of current in the loop antenna, while arrows in the clockwise direction represent the direction of parasitic current in the metallic layer 24.

FIG. 14 also shows the currents in a loop antenna arrangement with the parasitic currents. In the lower frequency arrangement (e.g., about 13.56 MHz), the NFC blocking element 46 (e.g., capacitor) is “open” and the loop antenna is effectively formed by the first and second antenna components 26, 28, surrounded by the parasitic element of the metallic layer 24.

FIG. 15 is a diagram illustrating an embodiment of the smart ring 12, which may further include a strip of conductor film 70 formed on one surface (e.g., inner surface) of the first antenna component 26 (e.g., battery casing). In this embodiment, the battery jacket may be at least partially conductive and the conductor film 70 may be used as a conductor for providing more predictable antenna properties, such as improving conductivity, reducing resistance, etc. Also, the conductor film 70 may be added over the first antenna component 26 from the first electrical circuit 30 to the high frequency choke inductor element 52.

FIG. 16 is a diagram illustrating another embodiment of the smart ring 12, which may further include first and second strips of ferrite sheets 74, 76, in addition to the strip of conductor film 70 shown in FIG. 15. The first strip of ferrite sheet 74 may be formed on a surface (e.g., inner surface) of the first antenna component 26 (e.g., battery casing), which may then be surrounded by the conductor film 70 in some embodiments. The second strip of ferrite sheet 76 may be formed on a surface (e.g., an outer surface) of the second antenna component 28 (e.g., FPC), such as between the metallic layer 24 and the FPC. If NFC antenna efficacy needs to be increased, one or more of the ferrite sheets 74, 76 can be placed on one or more of the first and second antenna components 26, 28.

FIG. 17 is a photograph showing a perspective side view of a Near-Field Communications (NFC) charger 80 configured for charging the smart ring 12 when place in proximity thereto. FIG. 18 is a photograph illustrating a top view of the NFC charger 80. The NFC charger 80 may include a coil 82, which may be embedded in a tubular post. When the smart ring 12 is placed over the post, NFC charger 80 is configured to cause the coil 82 to generate a magnetic field (e.g., similar to the magnetic field shown in FIG. 13). This magnetic field may be used to charge the battery of the smart ring 12.

The charger 80 induces currents in the antenna components 26, 28 that are larger than the currents that already exist in the smart ring 12. The additional of the ferrite sheets 74, 76 (FIG. 16) between the inner antenna component and the outer metallic layer 24 can be used to improve the performance of the NFC antenna (e.g., which may be possible at some expense of the performance at the higher frequency implementations). The smart ring 12 can be placed on the NFC charger 80 for charging. Since the smart ring 12 may be rotationally independent, it can be placed in any rotational or up/down position. Charging the NFC antenna component's magnetic field is configured to couple with the ring's magnetic field.

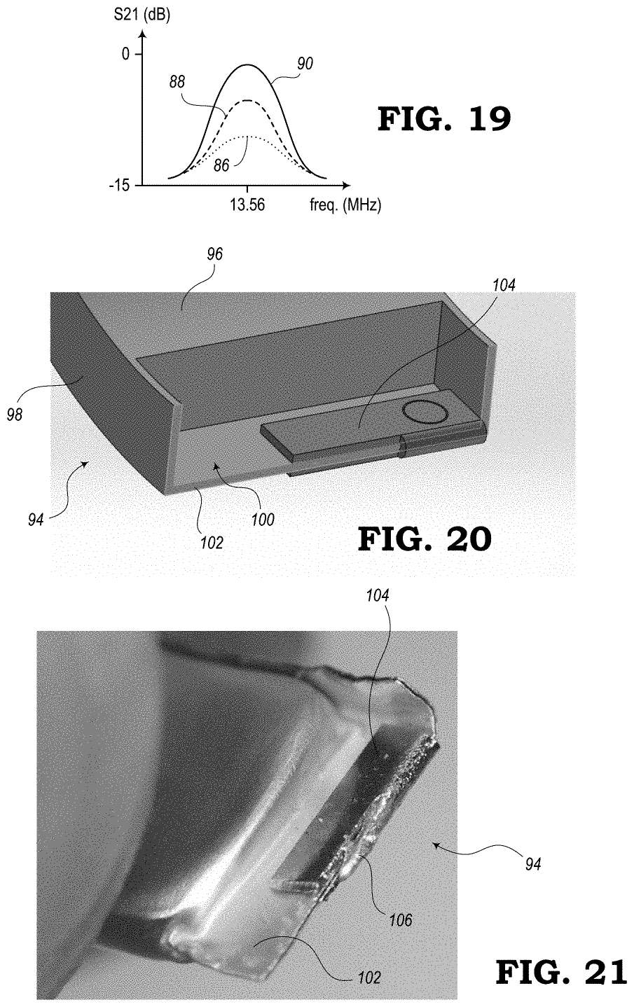

FIG. 19 is a graph illustrating S21 (e.g., S parameter related to forward voltage gain) versus frequency of the NFC charger 80 for operation within the second (lower) frequency band. The curve 86 may represent the response based on the configuration shown in FIG. 5. The curve 88 may represent the response based on the configuration shown in FIG. 15 with the strip of conductor film 70 added. Also, the curve 90 may represent the response based on the configuration shown in FIG. 16 with the conductor film 70 and ferrite sheets 74, 76 added. All three implementations work well for NFC payment applications with a suitable POS device. Also, all three implementations work well for wireless charging applications. The power transfer function from the charger 80 to the smart ring 12 (and vice versa) may include scattering parameters or S parameters (S11, S21, S12, S22), where S21 is shown in the graph of FIG. 19.

FIG. 20 is a diagram illustrating a perspective view of an end portion of a battery casing 94, which may be used as the first antenna component 26. In this view, FIG. 20 shows the battery casing 94 with an inner surface 96 (facing away from the metallic layer 24 within the smart ring 12) and a side portion 98 facing a top (or bottom) end of the smart ring 12. FIG. 21 is a photograph illustrating the end portion of the battery casing 94. In this embodiment, the battery casing 94 also includes a pouch 100 and an edge 102 (of the pouch 100 or battery casing 94). A metal clip 104 may be attached over at least a portion of the edge 102. The metal clip 104 can be attached by crimping the material over the edge 102, by soldering 106, and/or by other suitable ways to ensure a sufficient conductive contact.

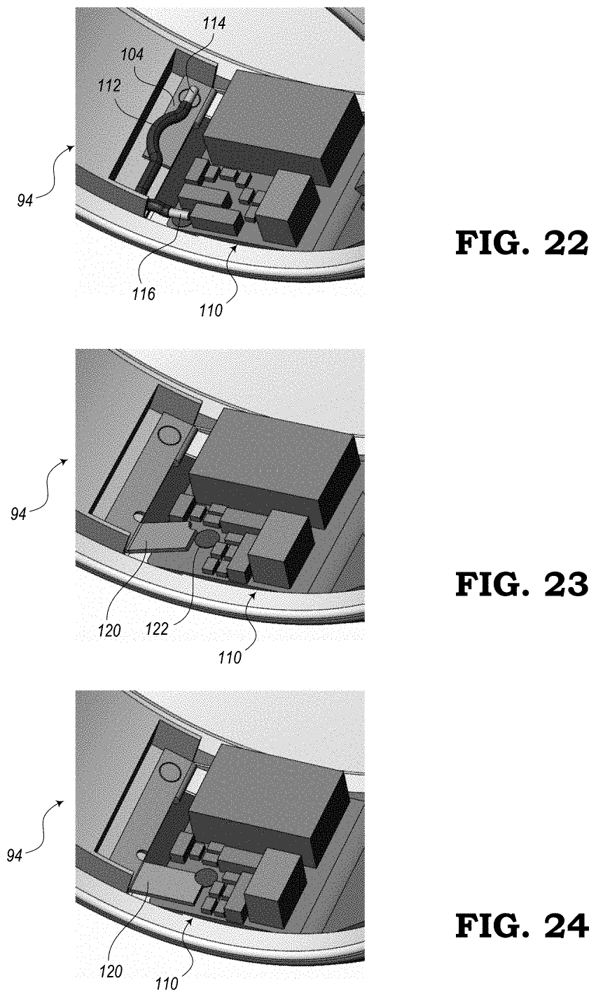

FIGS. 22-24 are diagrams illustrating connections between the battery casing 94 of FIGS. 20 and 21 and an end portion of a FPC 110, which may be used as the second antenna component 28. For example, the FPC 110 may include non-conductive board elements (flexible or rigid) on which electrical circuitry may reside. The electrical circuitry, for instance, may include the higher frequency elements 46, 48, 50 and the lower frequency elements 52, 54, 56, 58 shown in FIG. 3. In this respect, the electrical connection between the first and second antenna components 26, 28 may include the second electrical circuit 32, which may be housed on the FPC 110.

The connection between the battery casing 94 (or jacket) and the FPC 110 may include soldering ends of a wire 112 as shown in FIG. 22. A first end 114 of the wire 112 may be soldered to the metal clip 104 in the pouch 100 of the battery casing 94 and a second end 116 of the wire 112 may be soldered to a contact on the FPC 110. In some cases, a clip 120 (FIG. 23) may be soldered in place on the battery casing 94 and then used to force fit (e.g., pinch) a conductive contact 122 on the FPC 110 (FIG. 24).

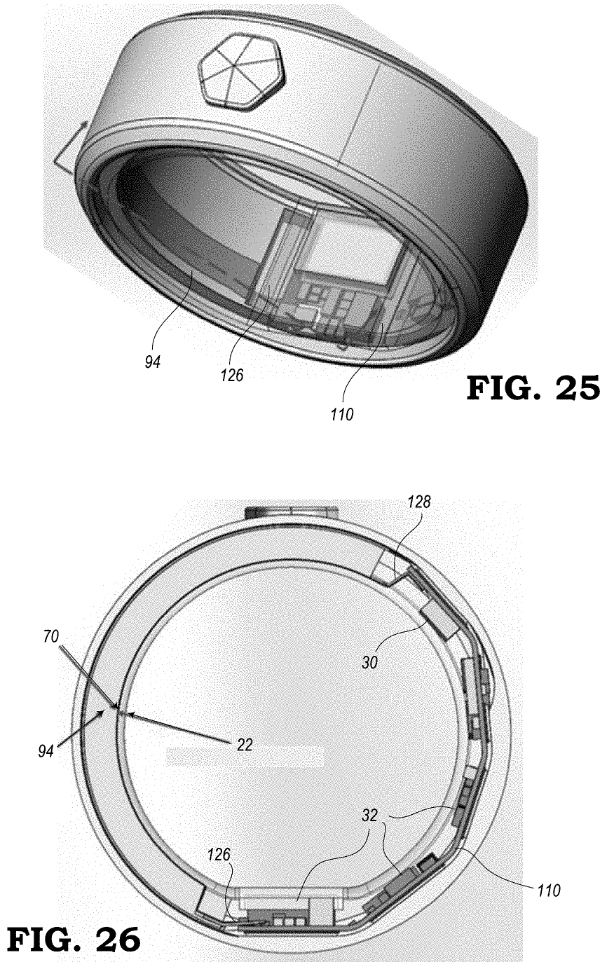

FIG. 25 is a diagram illustrating an isometric, partially cut-away view of the smart ring 12. Also, FIG. 26 is a diagram illustrating a cross-sectional view of the smart ring 12. The battery casing 94 is embedded in the smart ring 12 and is connected via a first set of connections 126 (e.g., one of the connection implementations described with respect to FIGS. 22-24) to the second electrical circuit 32 formed on the FPC 110. A second set of connections 128 is formed between the other end of the battery casing 94 and the other end of the FPC 110 and is used for connection to the first electrical circuit 30, which may also be formed on the FPC 110.

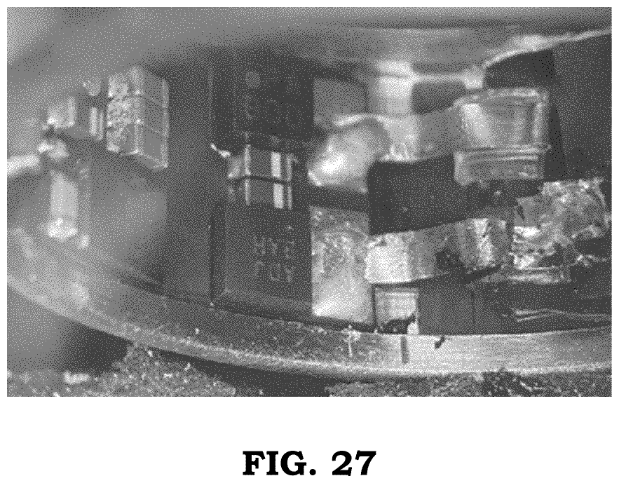

FIG. 27 is a photograph illustrating connections (e.g., second set of connections 128) from positive and negative terminals of a battery (e.g., protected by the battery casing 94) to the other end portion of the FPC 110. Using the positive (+) and negative (−) battery leads the natural capacitance, the connections 128 are configured for electrical contact between the battery (and/or battery case) and the FPC 110 to complete the circuit through the high frequency choke inductor 30. In some embodiments, a clip may be used at both ends of the battery to achieve the connection.

### Physical Aspects

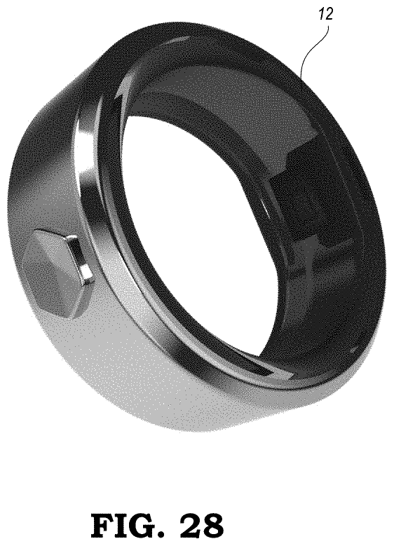

FIG. 28 is a diagram of a smart ring 12. The smart ring 12 can include a titanium finishing, light weight and slim profile (e.g., <3.5 mm thick, 8 mm width), water resistant, 1 week battery life, various sizes, etc. In an embodiment, the smart ring 12 can include various sensors such as a 14-bit Photoplethysmography (PPG) and 3-axis accelerometer. The smart ring 12 can be configured to measure vitals such as heart rate, heart rate variability, sleep, activity, fall, and the like.

The charging case can include a lid and looks/works a bit like a jewelry box. In an embodiment, the charging case can have no buttons, lights, or anything on the charger. The charging case can include a hexagonal shape.

For ring sizing, various approaches are contemplated such as a strap that user puts around their finger to measure size. Another approach can be plastic ring sizers for the user to try on—these can have a hexagonal shape. In another embodiment, there can be a ring sizer built into a mobile application that includes a template that shows up in real scale and you check which is most similar size to an existing ring. Another approach can utilize a camera on a mobile device to take a picture of an existing ring to determine its size. Also, it is possible to place the finger on the mobile device screen to compare to dimensions marked thereon.

To ensure the smart ring 12 always has battery charge, a user can be provided two smart rings 12 so that one can be worn while the other is charging. Data from both is automatically aggregated, and both can fit in charger and be charged at the same time.

### Distributed Wi-Fi System

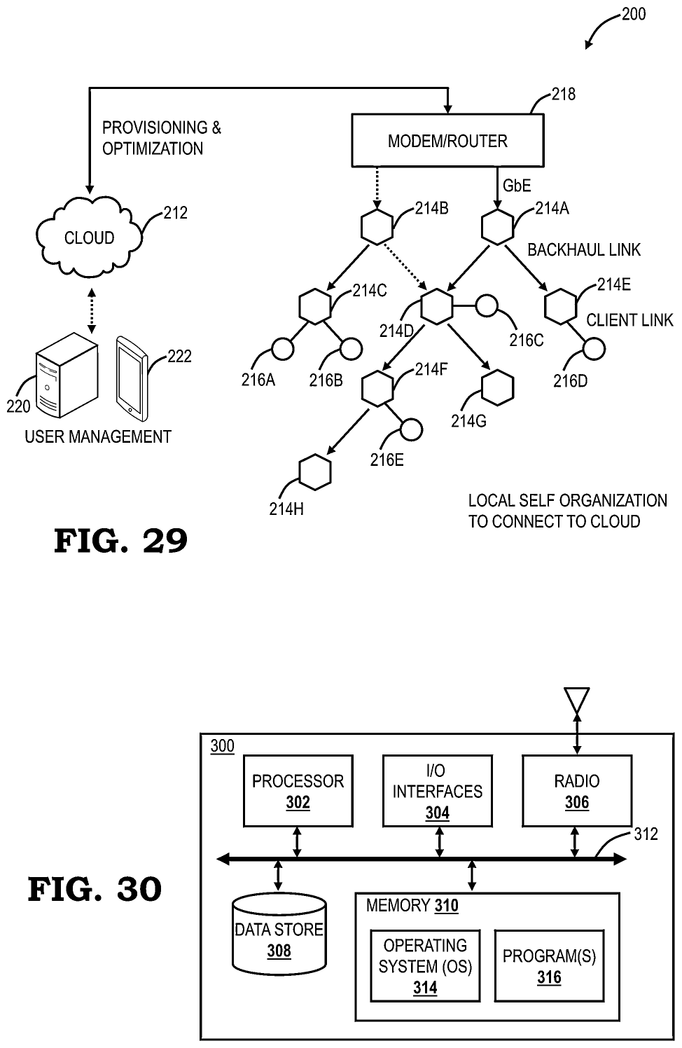

FIG. 29 is a network diagram of a distributed Wi-Fi system 200 with control via a cloud 212 service. The distributed Wi-Fi system 200 can operate in accordance with the IEEE 802.11 protocols and variations thereof. The distributed Wi-Fi system 200 includes a plurality of access points 214 (labeled as access points 214A-214H), which can be distributed throughout a location, such as a residence, office, or the like. That is, the distributed Wi-Fi system 200 contemplates operation in any physical location where it is inefficient or impractical to service with a single access point, repeaters, or a mesh system. As described herein, the distributed Wi-Fi system 200 can be referred to as a network, a system, a Wi-Fi network, a Wi-Fi system, a cloud-based system, etc. The access points 214 can be referred to as nodes, access points, Wi-Fi nodes, Wi-Fi access points, etc. The objective of the access points 214 is to provide network connectivity to Wi-Fi client devices 16 (labeled as Wi-Fi client devices 216A-216E). The Wi-Fi client devices 216 can be referred to as client devices, user devices, clients, Wi-Fi clients, Wi-Fi devices, etc.

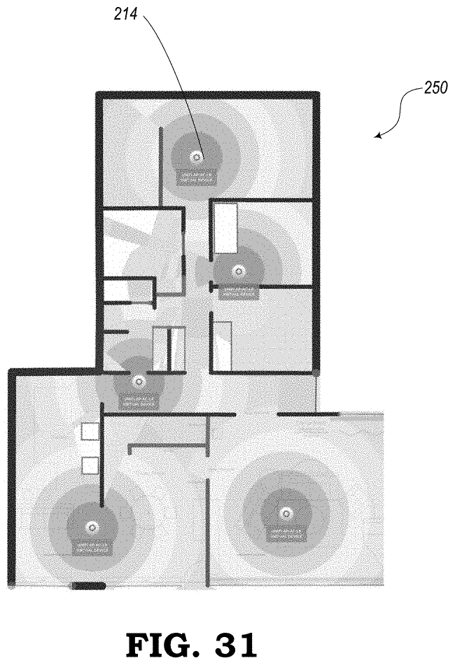

In a typical residential deployment, the distributed Wi-Fi system 200 can include between 3 to 12 access points or more in a home. For example, FIG. 31 is a diagram of a location 250 with access points 214 distributed therein, in different rooms. A large number of access points 214 (which can also be referred to as nodes in the distributed Wi-Fi system 200) ensures that the distance between any access point 214 is always small, as is the distance to any Wi-Fi client device 216 needing Wi-Fi service. That is, an objective of the distributed Wi-Fi system 200 can be for distances between the access points 214 to be of similar size as distances between the Wi-Fi client devices 216 and the associated access point 214. Such small distances ensure that every corner of a consumer's home is well covered by Wi-Fi signals. It also ensures that any given hop in the distributed Wi-Fi system 200 is short and goes through few walls. This results in very strong signal strengths for each hop in the distributed Wi-Fi system 200, allowing the use of high data rates, and providing robust operation. Note, those skilled in the art will recognize the Wi-Fi client devices 216 can be mobile devices, tablets, computers, consumer electronics, home entertainment devices, televisions, IoT devices, or any network-enabled device, including the smart ring 12. For external network connectivity, one or more of the access points 214 can be connected to a modem/router 218, which can be a cable modem, Digital Subscriber Loop (DSL) modem, or any device providing external network connectivity to the physical location associated with the distributed Wi-Fi system 200.

While providing excellent coverage, a large number of access points 214 (nodes) presents a coordination problem. Getting all the access points 214 configured correctly and communicating efficiently requires centralized control. This cloud 212 service can provide control via servers 20 that can be reached across the Internet and accessed remotely, such as through an application (“app”) running on a user device 222. The running of the distributed Wi-Fi system 200, therefore, becomes what is commonly known as a “cloud service.” The servers 220 are configured to receive measurement data, to analyze the measurement data, and to configure the access points 214 in the distributed Wi-Fi system 200 based thereon, through the cloud 212. The servers 220 can also be configured to determine which access point 214 each of the Wi-Fi client devices 216 connect (associate) with. That is, in an example aspect, the distributed Wi-Fi system 200 includes cloud-based control (with a cloud-based controller or cloud service in the cloud) to optimize, configure, and monitor the operation of the access points 214 and the Wi-Fi client devices 216. This cloud-based control is contrasted with a conventional operation that relies on a local configuration, such as by logging in locally to an access point. In the distributed Wi-Fi system 200, the control and optimization does not require local login to the access point 214, but rather the user device 222 (or a local Wi-Fi client device 216) communicating with the servers 220 in the cloud 212, such as via a disparate network (a different network than the distributed Wi-Fi system 200) (e.g., LTE, another Wi-Fi network, etc.).

The access points 214 can include both wireless links and wired links for connectivity. In the example of FIG. 29, the access point 214A has an example gigabit Ethernet (GbE) wired connection to the modem/router 218. Optionally, the access point 214B also has a wired connection to the modem/router 218, such as for redundancy or load balancing. Also, the access points 214A, 214B can have a wireless connection to the modem/router 218. The access points 214 can have wireless links for client connectivity (referred to as a client link) and for backhaul (referred to as a backhaul link). The distributed Wi-Fi system 200 differs from a conventional Wi-Fi mesh network in that the client links and the backhaul links do not necessarily share the same Wi-Fi channel, thereby reducing interference. That is, the access points 214 can support at least two Wi-Fi wireless channels—which can be used flexibly to serve either the client link or the backhaul link and may have at least one wired port for connectivity to the modem/router 218, or for connection to other devices. In the distributed Wi-Fi system 200, only a small subset of the access points 214 require direct connectivity to the modem/router 218 with the non-connected access points 214 communicating with the modem/router 218 through the backhaul links back to the connected access points 214.

One advantage of the distributed Wi-Fi system 200 is the access points 214 are distributed throughout a location, such as in different rooms. The access points 214 can be configured as a smart motion detection system that monitors family members or guests, when they arrive or leave, where they are located, etc. The smart motion detection system operates by detecting the disturbances in Wi-Fi signals between the access points 214 or between an access point 214 and a motion detection capable device. These disturbances in the signal are translated into motion events, which you can use to keep yourself aware of activity in your home.

Also, in an embodiment, the smart ring 12 can be configured to connect directly to one of the access points 214 in the distributed Wi-Fi system 200, in addition to connecting to a user device 300 (FIG. 30). Of note, the smart ring 12 can be used in conjunction with the smart motion detection system in the distributed Wi-Fi system 200. This can be for a variety of applications and use cases as described herein, including motion detection, safety (fall), geofencing for utilities, cadence tracking, reminders, security, etc.

### Example User Device Architecture

FIG. 30 is a block diagram of a user device 300, which may be used for the user device 222 or the like. The user device 300 can be a digital device that, in terms of hardware architecture, generally includes a processor 302, input/output (I/O) interfaces 304, a radio 306, a data store 308, and memory 310. It should be appreciated by those of ordinary skill in the art that FIG. 30 depicts the user device 300 in an oversimplified manner, and a practical embodiment may include additional components and suitably configured processing logic to support known or conventional operating features that are not described in detail herein. Also, the user device 300 may be referred to as a mobile device, smart phone, tablet, smart watch, etc. The components (302, 304, 306, 308, and 302) are communicatively coupled via a local interface 312. The local interface 312 can be, for example, but not limited to, one or more buses or other wired or wireless connections, as is known in the art. The local interface 312 can have additional elements, which are omitted for simplicity, such as controllers, buffers (caches), drivers, repeaters, and receivers, among many others, to enable communications. Further, the local interface 312 may include address, control, and/or data connections to enable appropriate communications among the aforementioned components.

The processor 302 is a hardware device for executing software instructions. The processor 302 can be any custom made or commercially available processor, a central processing unit (CPU), an auxiliary processor among several processors associated with the user device 300, a semiconductor-based microprocessor (in the form of a microchip or chipset), or generally any device for executing software instructions. When the user device 300 is in operation, the processor 302 is configured to execute software stored within the memory 310, to communicate data to and from the memory 310, and to generally control operations of the user device 300 pursuant to the software instructions. In an embodiment, the processor 302 may include a mobile optimized processor such as optimized for power consumption and mobile applications. The I/O interfaces 304 can be used to receive user input from and/or for providing system output. User input can be provided via, for example, a keypad, a touch screen, a scroll ball, a scroll bar, buttons, a barcode scanner, and the like. System output can be provided via a display device such as a liquid crystal display (LCD), touch screen, and the like. The I/O interfaces 304 can also include, for example, a serial port, a parallel port, a small computer system interface (SCSI), an infrared (IR) interface, a radio frequency (RF) interface, a universal serial bus (USB) interface, and the like. The I/O interfaces 304 can include a graphical user interface (GUI) that enables a user to interact with the user device 300. Additionally, the I/O interfaces 304 may further include an imaging device, i.e., camera, video camera, etc.

The radio 306 enables wireless communication to an external access device or network. Any number of suitable wireless data communication protocols, techniques, or methodologies can be supported by the radio 306. The data store 308 may be used to store data. The data store 308 may include any of volatile memory elements (e.g., random access memory (RAM, such as DRAM, SRAM, SDRAM, and the like)), nonvolatile memory elements (e.g., ROM, hard drive, tape, CDROM, and the like), and combinations thereof. Moreover, the data store 308 may incorporate electronic, magnetic, optical, and/or other types of storage media.

The memory 310 may include any of volatile memory elements (e.g., random access memory (RAM, such as DRAM, SRAM, SDRAM, etc.)), nonvolatile memory elements (e.g., ROM, hard drive, etc.), and combinations thereof. Moreover, the memory 310 may incorporate electronic, magnetic, optical, and/or other types of storage media. Note that the memory 310 may have a distributed architecture, where various components are situated remotely from one another but can be accessed by the processor 302. The software in memory 310 can include one or more software programs, each of which includes an ordered listing of executable instructions for implementing logical functions. In the example of FIG. 3, the software in the memory 310 includes a suitable operating system (O/S) 314 and programs 316. The operating system 314 essentially controls the execution of other computer programs and provides scheduling, input-output control, file and data management, memory management, and communication control and related services. The programs 316 may include various applications, add-ons, etc. configured to provide end-user functionality with the user device 300. For example, example programs 316 may include, but not limited to, a web browser, social networking applications, streaming media applications, games, mapping and location applications, electronic mail applications, financial applications, and the like. In a typical example, the end user typically uses one or more of the programs 316 along with a network.

### Mobile Application

In an embodiment, the programs 316 can include an application that manages the distributed Wi-Fi system 200 as well as the smart ring 12. This application can work with the cloud 212 to leverage active data collection and Artificial Intelligence (AI) to deliver insights to address challenges, such as related to personal wellbeing, activity, cadence, etc. as well as adaptive Wi-Fi, cybersecurity, access control, and motion detection via the distributed Wi-Fi system. The personal wellbeing can include stress management, active minutes, sleep management, fall detection, etc. The activity can include detecting suspicious activity, motion detection, etc. The cadence can include routine tracking, home automation, fall detection, coordination with heating and air conditioning based on location, sleep, and the like. Of note, individually each of these aspects targets a specific need, but together, richer data enables more accurate and holistically life management.

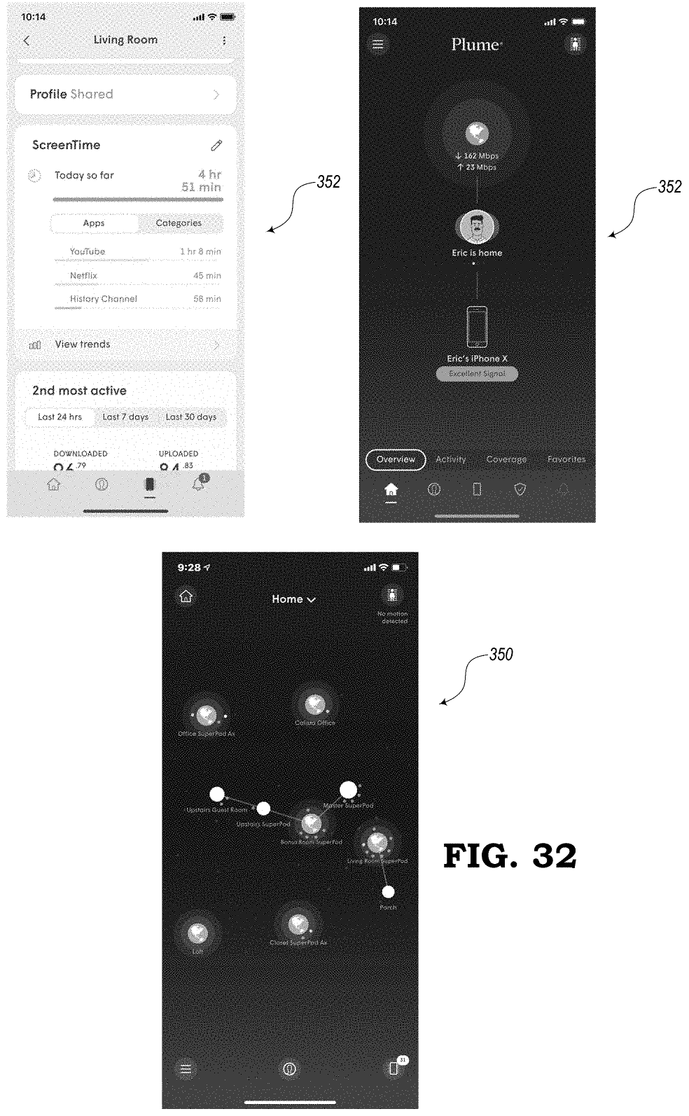

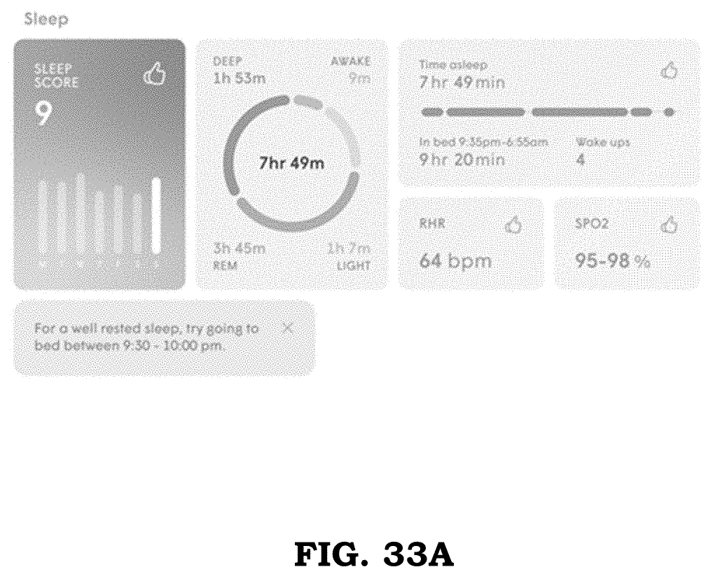

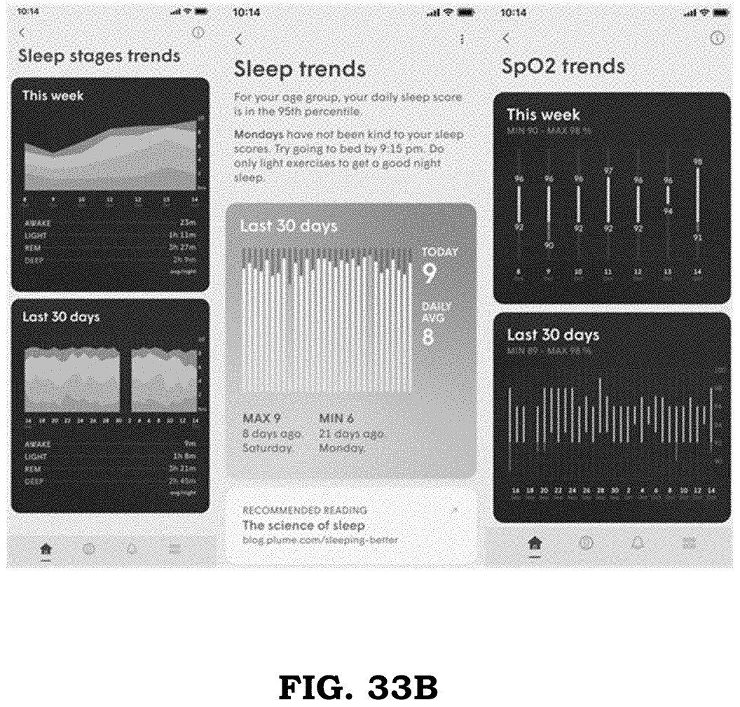

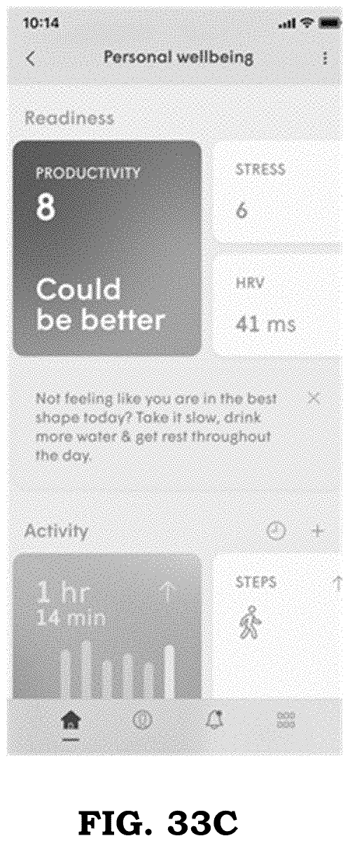

FIG. 32 is screenshots of a Graphical User Interface (GUI) from the application illustrating management of the distributed Wi-Fi system 200. FIGS. 33A, 33B, 33C are screenshots of the GUI from the application illustrating management of the smart ring 12. Advantageously, the application can combine management off the distributed Wi-Fi system 200 and the smart ring 12 in a single GUI. In FIG. 32, the application can include a network topology view 350, a device connectivity view 352, and a room view 354. The network topology view 350 shows access points 214 and orbiting dots representing client devices 216. The device connectivity view 352 includes a single device 216, its connectivity and speed. The room view 354 can include activity therein, active devices, etc. There can be other views for the smart motion detection system and the like.

In FIGS. 33A, 33B, 33C, the application can include an activity view 362, trend notifications 364, and graphs. The application, for the smart ring 12, can be a personal health coach that monitors your health and gives you personalized recommendations to stay healthy, in conjunction with the smart ring 12, the distributed Wi-Fi system 200, and the user device 300. This can provide AI-driven health monitoring. The application can support Fall detection, Sleep trend and stress detection via Heart Rate Variability (HRV), Blood oxygen trends, Activity and over activity monitoring, Heart rate monitoring, Care group notification, Blood pressure monitoring, body temperature, stress, and the like.

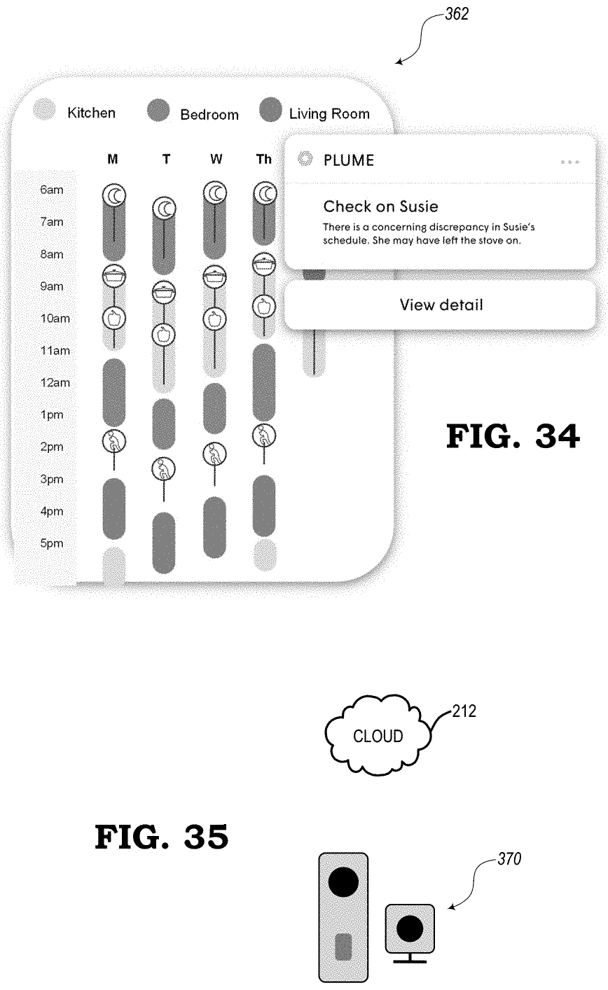

FIG. 34 is a screenshot of the GUI from the application illustrating location and motion detection. Here, a combination of the smart ring 12, the distributed Wi-Fi system 200, and the user device 300 can be used to track location, motion, and the like versus time. The idea here is to use the rich data from all of these devices 12, 200, 300 to detect a user's cadence. This can be confined to the home as well as external locations. For example, parents can track children's locations, loved ones can monitor elderly parents, and the like. This can be used for geo-fencing, e.g., turn on/off lights, heating and air, lock doors, monitor water safety (pools, ocean), etc. It is possible, with the cloud 212 service to monitor fall risk, daily routine and inconsistency alerts, interaction with Internet of Things (IoT) devices (left the stove on, refrigerator is open, etc.), and the like

### Third-Party Device Integration

FIG. 35 is a diagram of integration of third-party devices 370 with the cloud 212 service. For example, different vendors can make different devices including other smart rings, user devices, IoT devices, Wi-Fi devices, and the like. However, it is possible for unified control via the cloud using standardized techniques for communication with the cloud 212. One such example includes OpenSync, sponsored by the Applicant of the present disclosure and described at www.opensync.io/documentation. OpenSync is cloud-agnostic open-source software for the delivery, curation, and management of services for the modern home. That is, this provides standardization of the communication between devices and the cloud 212. OpenSync acts as silicon, Customer Premises Equipment (CPE), and cloud-agnostic connection between the in-home hardware devices and the cloud 212. This can be used to collect measurements and statistics from the connected Wi-Fi client devices 216 and network management elements, and to enable customized connectivity services.

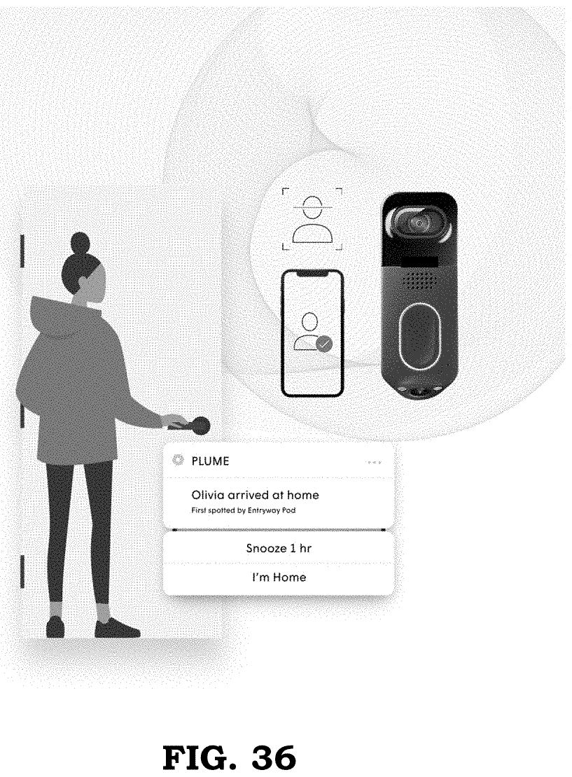

One application can include preventative surveillance leveraging OpenSync enabled cameras and the cloud 212 service's ability to understand situational context and intelligently alert you when necessary and record events that matter. For example, camera systems connected to the distributed Wi-Fi system 200 can be used for facial recognition—get notified about who is at your door and get alerted of anomalies. With the smart motion detection, there can be smart motion that customizes areas for your camera to focus on to eliminate busy areas that could cause false positives. The application can provide contextual notification—only record events that matter to save storage space and save you from having to weed through hours of irrelevant footage. FIG. 36 is a diagram of a use case where a doorbell camera is integrated with the cloud 212 service and the application to alert a specific person is at the door. Other applications can include motion zones, package detection, person detection, etc.

### Cloud Service AI

The following table illustrates example features and functions of a cloud 212 service with the smart ring 12, the distributed Wi-Fi system 200, and the user device 300.

| | Actions | Health Management | Home Automation |
|---|---|---|---|
| Insight | Automated action | Predictions to | Personalized home |
| | based on the object | actionable | automations e.g., |
| | and activity e.g., | recommendations | Turn on dim lights |
| | suspicious activity | e.g., sleep | toward bathroom |
| | alert | recommendations | |
| Predic- | Camera data to | Vitals data to | Activities, routine |
| tive | object and people | health trends and | and user |
| | recognition e.g., | predictions— | preferences— |
| | facial recognition | e.g., light/deep | e.g., movement |
| | | sleep hours | during sleep hours |
| Data | Raw image footage | Raw value to vitals | Presence and sensor |
| | e.g., video feed | data e.g., HRV | data e.g., |
| | | | movement |

The cloud 212 service can leverage a large amount of data and use this for cohort statistics to determine averages, trends, etc. for a given population. The cohort can be age, sex, health condition, fitness level, etc. This data can be anonymized and used to compare to an average situated cohort.

### Smart Ring Features

Again, the smart ring 12 contemplates use with the distributed Wi-Fi system 200, the user device 300, and the like as well as with the cloud 212 service. The smart ring 12 can support push notifications, provide indicators on the user device 300 such as related to connectivity, battery status, etc. With the cloud 212 service, there can be a fusion of data taken from the smart ring 12 and data taken from the distributed Wi-Fi system 200, the user device 300, and third-party devices 370. Home data can include network usage by time of day, applications used, types of devices used, and the like. By fusion, the cloud 212 service can provide analysis, for cadence and trends. For example, see worse HRV, note that person has been staying up very late using a gaming platform, send recommendation to cut back on gaming late at night.

The smart ring 12 can conserve its battery in a variety of ways. First, the data movement to the cloud 212 or the user device 300 can be only when triggered (polled) when the application is open on the user device 300. Alternatively, the data can be pushed in case a storage threshold is reached. The smart ring 12 includes memory for storing the various measurements. Also, the smart ring 12 can include an accelerometer to wake up processing or communication only when there is a change in the physical motion of the ring 12.

The smart ring 12 can include a Bluetooth antenna as described herein and associated circuitry and software for connectivity therewith. In an embodiment, the smart ring 12 can communicate with the access points 214 and use this information to determine location and create maps of where a wearer typically spends time in the location 250. There can be various use cases, such as, counting bathroom trips, counting kitchen trips, counting time spent in bed, and the like. The Bluetooth antenna can work with the Bluetooth or Wi-Fi on the access points 214.

### Use Cases

The smart ring 12 includes a battery, memory, processing circuitry, a plurality of sensors, a plurality of antennas, and a battery, all enclosed in a casing (e.g., titanium). These components work together for connectivity to external devices, namely the cloud 212 service, the distributed Wi-Fi system 200, the user device 300, third-party devices 370, Point-of-Sale (PoS) devices, and the like. The following are non-limiting use cases with the smart ring 12 and other components.

Measurement of SPO2 (oxygen saturation) via a PPG sensor that looks at wavelengths that are absorbed, reflected from blood flow in the finger.

Device syncing—traditional rings sync with a phone. The smart ring 12 contemplates synchronizing with multiple devices, namely the access points 214 as well as the user device 300. This feature is useful as a user's phone may be in a different room, not nearby, and the smart ring 12 can sync to a closer access point 214, via either Wi-Fi or Bluetooth.

Information sharing—the smart ring 12, via the cloud 212 service, can share information with third parties, such as, nominating someone (e.g., child, parent, caregiving, health care workers, etc.). The wearer can set up permission as well as a guardian. This information sharing can be for location tracking, fall detection, health monitoring, geo-fencing, etc.

Defining activity with heart rate zones—it is possible to measure how long people are spending in each heart rate zone to ensure sufficient aerobic activity in a week.

Content partners—it is possible to pass information to a content partner for integration with the smart ring 12. Non-limiting examples can include meditation for better sleep or lower stress, breathing exercises, yoga, diet, etc. The content supplier can be a third party. Also, the user can opt in or opt out of the third-party contact.

Elderly—the smart ring 12 can be used for wellbeing, e.g., fall detection, sharing with caregivers, the ring that can make emergency contact based on a microphone or tap sensor (e.g., specific sequence) or automatically based on event detection (fall).

Ring input/output (I/O)—the smart ring 12 can include vibration/haptic feedback, a tiny speaker, a microphone, a sensor for tap patterns, etc.

Thresholds—the various sensors in the smart ring 12 can be used to alert when various thresholds are detected, such as SPO2 min, Heart rate max, fall, etc. The notification can include push notifications, third-party notifications (caregiver, guardian, etc.), via the application on the user device 300, an audible alarm for thresholds, a visual indicator, a haptic/vibration indicator, etc.

### CONCLUSION

It will be appreciated that some embodiments described herein may include or utilize one or more generic or specialized processors (“one or more processors”) such as microprocessors; Central Processing Units (CPUs); Digital Signal Processors (DSPs): customized processors such as Network Processors (NPs) or Network Processing Units (NPUs), Graphics Processing Units (GPUs), or the like; Field-Programmable Gate Arrays (FPGAs); and the like along with unique stored program instructions (including both software and firmware) for control thereof to implement, in conjunction with certain non-processor circuits, some, most, or all of the functions of the methods and/or systems described herein. Alternatively, some or all functions may be implemented by a state machine that has no stored program instructions, or in one or more Application-Specific Integrated Circuits (ASICs), in which each function or some combinations of certain of the functions are implemented as custom logic or circuitry. Of course, a combination of the aforementioned approaches may be used. For some of the embodiments described herein, a corresponding device in hardware and optionally with software, firmware, and a combination thereof can be referred to as “circuitry configured to,” “logic configured to,” etc. perform a set of operations, steps, methods, processes, algorithms, functions, techniques, etc. on digital and/or analog signals as described herein for the various embodiments.

Moreover, some embodiments may include a non-transitory computer-readable medium having instructions stored thereon for programming a computer, server, appliance, device, at least one processor, circuit/circuitry, etc. to perform functions as described and claimed herein. Examples of such non-transitory computer-readable medium include, but are not limited to, a hard disk, an optical storage device, a magnetic storage device, a Read-Only Memory (ROM), a Programmable ROM (PROM), an Erasable PROM (EPROM), an Electrically EPROM (EEPROM), Flash memory, and the like. When stored in the non-transitory computer-readable medium, software can include instructions executable by one or more processors (e.g., any type of programmable circuitry or logic) that, in response to such execution, cause the one or more processors to perform a set of operations, steps, methods, processes, algorithms, functions, techniques, etc. as described herein for the various embodiments.

Although the present disclosure has been illustrated and described herein with reference to preferred embodiments and specific examples thereof, it will be readily apparent to those of ordinary skill in the art that other embodiments and examples may perform similar functions and/or achieve like results. All such equivalent embodiments and examples are within the spirit and scope of the present disclosure, are contemplated thereby, and are intended to be covered by the following claims. Moreover, it is noted that the various elements, operations, steps, methods, processes, algorithms, functions, techniques, etc. described herein can be used in any and all combinations with each other.

What is claimed is:

1. A smart ring comprising:
a battery, memory, processing circuitry, a plurality of sensors, a plurality of antennas, and a battery, each coupled to one another and all enclosed in a casing,
wherein the processing circuitry is configured to conserve the battery by any of sending data to the cloud service when an application is open on the user device, sending data to the cloud service when a threshold is crossed, waking up processing or communicating when there is a change in motion detected by the accelerometer.

2. The smart ring of claim 1, wherein the processing circuitry is further configured to
cause the smart ring to pair with any of a user device and a Wi-Fi access point in a distributed Wi-Fi system, and
obtain measurements from any of the plurality of sensors and provide the measurements to a cloud service, via the any of the user device and the Wi-Fi access point.

3. The smart ring of claim 1, wherein the plurality of antennas include an antenna for Bluetooth and Wi-Fi and an antenna for near field communication.

4. The smart ring of claim 1, wherein the plurality of antennas include an antenna for Bluetooth, wherein the antenna for Bluetooth is utilized in combination with the distributed Wi-Fi system to track a location of a wearer.

5. The smart ring of claim 4 wherein the tracking of location of a wearer is used for one or more of counting bathroom trips, counting kitchen trips, counting time spent in bed.

6. The smart ring of claim 1, wherein the plurality of sensors include a Photoplethysmography (PPG) sensor and an accelerometer.

7. The smart ring of claim 6, wherein the PPG sensor is configured to measure oxygen saturation.

8. The smart ring of claim 6, wherein the accelerometer is configured to detect motion and falls.

9. The smart ring of claim 1, wherein the ring includes a plurality of a microphone, a speaker, haptic feedback, or a tap sensor.

10. The smart ring of claim 9, wherein the ring is configured too contact caregivers or emergency personnel based on input from one or more of the microphone or tap sensor.

11. A cloud system comprising:
one or more processing devices comprising processors and memory storing instructions that, when executed, cause the processors to
communicate with a smart ring containing a plurality of sensors and a plurality of antennas for measurements, wherein the communication with the smart ring saves power on the smart ring by communicating only when one or more of an application is open on a user device, a threshold is crossed, or there is a change in motion detected by an accelerometer in the ring.

12. The cloud system of claim 11, wherein the instructions that, when executed, further cause the processors to
communicate with any of a user device and a Wi-Fi access point in a distributed Wi-Fi system, the any of the user device and the Wi-Fi access point is paired to the smart ring, and
obtain the measurements from the memory in the smart ring, via the any of the user device and the Wi-Fi access point.

13. The cloud system of claim 11 wherein the instructions that, when executed, further cause the processors to
aggregate data from an at least first and second smart ring, such that a wearer wears one of the smart ring and the second smart ring at a time while another is charged.

14. The cloud system of claim 11, wherein the measurements are combined with other measurements taken by the distributed Wi-Fi system.

15. The cloud system of claim 14, wherein the other measurements include one or more of network usage, application usage, types of devices used.

16. The cloud system of claim 11, wherein the communication with the ring includes data from one or more of an accelerometer, a Photoplethysmography (PPG) sensor, a microphone, or a tap sensor.

17. The cloud system of claim 11, wherein the plurality of antennas include an antenna for Bluetooth, wherein the antenna for Bluetooth is utilized in combination with a distributed Wi-Fi system including the Wi-Fi access point to track a location of a wearer.

18. The cloud system of claim 11, wherein the instructions that, when executed, cause the processors to
combine the measurements with cohorts for comparison thereof.

19. The cloud system of claim 11, wherein the instructions that, when executed, cause the processors to
provide a notification to a third party based on a threshold being crossed.

20. The cloud system of claim 11, wherein the instructions that, when executed, cause the processors to
track a routine of a wearer based on the measurements.

21. The cloud system of claim 11, wherein the instructions that, when executed, cause the processors to
provide a Graphic User Interface (GUI) to display trends related to the measurements.

22. The cloud system of claim 11, wherein the instructions that, when executed, cause the processors to
detect issues based on the measurements and present notifications based thereon.

23. The cloud system of claim 11, wherein a portion of the data communicated is provided to a third party that supplies third party services.

24. The cloud system of claim 23, wherein the third party services includes one or more of Meditation, Breath Training, Yoga, Diet, Exercise, Counseling, Healthcare.
